SEQUENCE LISTING (36 sequences)

SEQ ID NO: 1 - protein, 530 aa, Enterococcus faecium
MKKSLISAVMVCSMTLTAVASPIAAAADDFDSQIQQQDQKIADLKNQQADAQSQIDALESQVSEINTQAQDLLAKQDTLRQESAQLVKDIADLQERIEKREDTIQKQAREAQVSNTSSNYIDAVLNADSLADAIGRVQAMTTMVKANNDLMEQQKQDKKAVEDKKAENDAKLKELAENQAALESQKGDLLSKQADLNVLKTSLAAEQATAEDKKADLNRQKAEAEAEQARIREQQRLAEQARQQAAQEKAEKEAREQAEAEAQATQASSTAQSSASEESSAAQSSTTEESSSAAQSSTTEESTTAPESSTTEESTTAPESSTTEESTTVPESSTTEESTTVPESSTTEESTTVPESSTTEESTTVPETSTEESTTPAPTTPSTDQSVDPGNSTGSNATNNTTNTTPTPTPSGSVNGAAIVAEAYKYIGTPYVWGGKDPSGFDCSGFTRYVYLQVTGRDIGGWTVPQESAGTKISVSQAKAGDLLFWGSPGGTYHVAIALGGGQYIHAPQPGESVKVGSVQWFAPDFAVSM

SEQ ID NO: 2 - protein, 518 aa, Enterococcus durans
MKKSLISAVMLSSIALTAVGSPIAAAADDFDSQIQQQDQKIADLQNQQASAQSQIEALEGQVSSINAKAQDLLTKQDTLRKESSQLEKEIADLQERIEKREATIQKQARETQVKNTSSNYIDAVLNADSLADAVGRIQAMTSIVKANQDLVDQQKQDKQAVEDKKAENEAKQKELSANQATLESQKGDLLAKQADLNVLKTSLAAEQATAEDKKADLNRKKAEAEAEQARIREQARLAEQARQQAAQEKAEKEAREQAAAQAAQSQAAQSQAASSASTTENSSTVQSSTTENSSSSAQSSSSSSSAVVTPGSSSTTEESTVPESSTSTTENSSTESSTTDSSVTESTTVPESSTQETTPATPTTPSTPATSNNGSTGNGGTPNNTGTVVTPPTTPSTPSGSVNGSAIVAEAYKYIGVPYVWGGKDPSGFDCSGFTSYVYKQVTGRDIGGWTVPQENAGAKISVSQAKAGDLLFWGSPGGTYHVAIALGGGQYIHAPQPGESVKVGSVQWFAPDFAVSM

SEQ ID NO: 3 - protein, 516 aa, Enterococcus hirae
MKKSLLSAVMLSSIALTAVGSPIAAAADDFDSQIQQQDKKIADLQNQQASAQSQIEALEGQVSAINTKAQDLLTKQDTLRKESAQLKQEIKDLQERIEKREATIQKQARETQVKNTSSNYIDAVLNADSLADAVGRIQAMSTIVKANQDLVQQQKEDKQAVEAKKAENEAKQKELADNQAALESQKGDLLAKQADLNVLKTSLAAEQATAEDKKADLNRKKAEAEAEQARIREQARLAEQARQQAAQEKAEKEAREQAAAQAAQTQALSSASTTTESSSAAQSSSEESKAPESSTTEESTSTESSTTTENSSTGSSSTESSSTEESTVPESSTQESTPANTESSSSSSNTNVNNNTNNSTNNSTNNSTTNNNNNNNTVTPAPTPTPTPAPAPAPNPSGSVNGAAIVAEAYKYIGTPYVWGGKDPSGFDCSGFTRYVYLQVTGRDIGGWTVPQESAGTKISVSQAKAGDLLFWGSAGGTYHVAISLGGGQYIHAPQPGENVKVGSVQWYTPDFAVSM

SEQ ID NO: 4 - protein, 500 aa, Enterococcus mundtii
MKKSLISAVMVSSMALTAVASPIAAAAEDFDSQIQQQDQKIAELQNQQASAQSQIEALEGQVADINTKAETLLANQATLRQESSQLTQEIADLQERIEKREATIQEQARETQVKGTSSNYIDAVLNAESFSDAIGRVQAMSSIVRANQDLVKQQKEDKQAVEDKKAENEAKLQELAENQAALESQKGDLLSKQADLNVLKTTLAAEQATAEDKKEDLNRQKAEAEAEQARIREQARLAEQARQQAAQEQAEREAREQAAVAAAAAQEQEQASSSSVQESTEVSESATSESSSSAESSTEQSSVPESSTSTEDSTTESSVPESSTEESTTTPSVPETTTPSTPEPSTPAPSTPEPSTPAPSTPAPSTPEPSTPAPSIPAPTAPSTNGAAIVAEAMKYIGTPYVWGGKDPSGFDCSGFTRYVYLQVTGRDIGGWTVPQESAGARISVSQAKAGDLLFWGAAGGTYHVAISLGGGQYIHAPQPGESVKIGSVQWYAPDFAVSM

SEQ ID NO: 5 - protein, 484 aa, Enterococcus raffinosus
MKKSLLSALMVCSVTLSALAAPAAASADEFDSKIADQDAKISSLQNQQTDAQSQISALEGEVASINEKAQGLLNEQASLRQESQDLQKQIETLQKRIEKRSEAIKEQARDTQVKQSSGTNVIDVVLNAESFTDAVSRVQAMTTIVKANNDLVEQQKADKAEVEQKQAENKKQQEQIAANQATLESQKGELITKQADLNVQTTTLAAEQATAEGEKASLKEKQEAAIKEQERVQEEARKAAEAQEAAKKADADAKAKADADAKAEADRKAQEEAAASTTTTESSTVESSSTVESSTTEQQTQSSATESSSTASSSEDNFQGGGATPTPSTTEDSGSSNQGSTSSSTNNNQTPSTPTPAPTPTPAPAPSGNTGGVVAEAMKYIGTPYVWGGKTPAGFDCSGFTSYVYRQATGREIGGWTVPQESAGTRISVDQAQAGDLYFWGSPGGSYHVAIAMGGGQYIHAPQPGETVTVSSVSYYAPSFAVRM

SEQ ID NO: 6 - protein, 484 aa, Enterococcus gilvus
MKKSLLSALMVCSVTLSALAAPAAASADEFDSKIADQDAKISSLQNQQTDAQSQISALEGEVASINDKAQGLLNEQASLKQKSQELQKQIETLDKRIEKRSEAIKEQARSAQVKQSSGSNVVDVVLNAESFTDVVSRVQAMATIAKANNDLVEQQKADKAEVEQKQSENQKQLEQIAANQATLESQKGDLITKQADLNVQTTTLAADQATAEGEKASLKEKQDAAIKEQQRVQEEARKAAEAQEAAKKAETDAKAKADADAKAEADRKAQEEAAASTTTTESSTVESSTTVETSSTEQQTSTTESSSSESSSEDNFQGGGATPTDNGNSSANNNQGSTSSSTNDQTPAPTPTPAPEVPKPSTPTPPPATSGSVVAEAMKYIGTPYVWGGKTPAGFDCSGFTSYVFRQATGREIGGWTVPQESAGTRISVGEAQAGDLYFWGSPGGTYHVAIAMGGGQYIHAPQPGETVTVSSVSYYAPSFAVRM

SEQ ID NO: 7 - protein, 505 aa, Enterococcus villorum
MKKSLLSAVMLSSIALTAVGSPIAAAADDFDSQIQQQDKKIADLQNQQLSAQSQIEALEGQVSAINTKAQDLLAKQDTLRKESTQLKKEIADLQERIEKREATIQKQARETQVKNTSSNYIDAVLNADSLADAVGRIQAMSSIVKANQDLVQQQKEDKQAVEAKKAENEAKQKELAENQAALESQKGDLLAKQADLNVLKTSLAAEQATAEDKKADLNRKKAEAEAEQARIREQARLAEQARQQAAQEKAEKEAREQAAAQAALSQATSTTESSSTVSSSTTESSSVAQSSSEESTTSESSTTTTEESTTSESSTTTTEESTVPESSTTTENSSTDSSTTESSVTESSTVPESSTQDSTTSTNTSNSNNSNNATTPTTPSTPSTPSGSVNGAAIVAEAYKYIGTPYVWGGKDPSGFDCSGFTRYVYLQVTGRDIGGWTVPQESAGAKISVSQAKAGDLLFWGSPGGTYHVAIALGGGQYIHAPQPGENVKVGSVQWYAPDFAVSM

SEQ ID NO: 8 - protein, 509 aa, Enterococcus ratti
MKKSLLSAVMLSSIALTAVGSPIAAAAEDFDSQIQQQDKKIADLQNQQSSAQAQIEALEDQVSTINAQAQDLLAKQATLRKESAQLKQEIADLQERIEKREATIQKQARETQVKNTSSNYIDAVLNADSFADAIGRIQAMSSIVKANQELVQQQKEDKQAVEAKKNENEAKQKELAKNQAVLESQKGDLLAKQADLNVLKTSLAAEQATAEGKKAELNRKKAEAQAEQARIREQARLAEQARQQAAREKAEKEAREQAAAQTKASTTEDSSVAQSSSQESAASESSTTSTEGSSTANSSTTAENSSTGSSSSESSVTDESTISDSSTSDSTPATDSGSSNSSSSNNAGNTSDSSSDSSNPTGDSSTSNDTNNTNNSDNAVVPTPAPSQPAGSVNGSSIVAEAYKYIGVPYVWGGKDPSGFDCSGFTRYVYLQATGRDIGGWTVPQESAGTRISVSQAKAGDLLFWGSPGGSYHVAISLGGGQYIHAPQPGESVKVGSVQWFAPDFAVSM

SEQ ID NO: 9 - protein, 422 aa, Enterococcus cecorum
MVKKRLSSVVIVSTVLAGTLVAPIATFADNYDSQIEQKNSEINDLKSKQSEAQDQIDRLETSINKINKKADELLKEQSTLREETVQLQKDIEVLTERIAKREEAIRNQARDVQVNNQSSVYVKALLDATSFTDALGRLKAMTTIVNANNDLVNQQKADKKAVEDKKAENEAKQEEIAKNQATLEEQKGTLEAKQADLNVLKASLAEQQATKESEKQALAEQKAAFEAEQKRVREQQAQAAAVQQAAQQAQASASTSSNAAASTNSNAGSSSSQASSSNSASSNASSSNAGVSNVVIPSRPAPAPSGNGSAIVAEAYKHIGKPYVWGAKGPNTFDCSGFTRYVYLQVTGRDIGGWTVPQEGAGAIIPVSQAQPGDLYFWGSRGSSYHVAIALGGGSYIHAPQPGESVKVGSVAYFAPSFAVRM

SEQ ID NO: 10 - protein, 436 aa, Enterococcus phoeniculicola
MKKSLLSTVMICSLTLTTLASPLVATADNLDDQIAQQNQKISELQGQQADVQAQISSLQAEVDTINGKAEDLLAKQKELYTKSDELKTEIKNLQERIEKREEAITDQARDVQVNGGSSNFIDAVLNADSFTDAIGRVQAMNTIVQANNDLVEQQKQDKADVEAKEAENKKQLEEIAANQAELENQKGVLTEKQADLNVLTTTLAAEQATAEGKKSDLNKQKEAAIAEQARVQAEAKKAEEVKAAAAAEKEKAEAEQAVTPVTETQTSGNTQNVVNNDEPAKVPETPAANSNTTSNTTPNTTPDTTPAEEKPVTPTPAPSGNGSSVVAEAYKYIGTPYVWGGKDPSGFDCSGFTSYVYRQATGREIGGWTVPQESAGAKIGINEAQAGDLLFWGSPGGTHHVAIALGGGQYIHAPQPGESVKIGSYQWYAPDFAVRM

SEQ ID NO: 11 - protein, 488 aa, Enterococcus saccharolyticus
MKKRVLTALLTCSLTLTAVAAPVAVFADDFDQQIEQKNKEISDLQAQQASIQDQISSLEGQISDINTKAEELIAKQQELAAQSQKLQEEIADLEVRIEKREEAIRKQARDVQVNGSDSNLVEAVLNADSLTDAIGRVQAMSTIVNANNELVNQQKEDKKAVETKKAENEAKQQEIAENQTALEAQKGEIQRSQADLDYLKADLALQQSSKEDEKKGIQKRKAEAEAERARIAEQERLAELARKAAAEAAAKQAQVEKEAQEAAKEQQAQVSSQEQVQQSTPATEAVAESTTEAPVANATSEEPAAVETPAETNQNTTQDTPTSTPVVEEKTVESTPVQEVVETPTVETPAPVEQTPVVETPKVETPKVESSAPTGSVVAEAYKYIGVPYVWGGKDPSGFDCSGFTSYVYRKATGREIGGWTVPQESAGSVISVSEAKAGDLLFWGSQGSTYHVAIALGGGQYIHAPAPGQSVTVASVAYFAPSFAVSM

SEQ ID NO: 12 - protein, 403 aa, Enterococcus columbae
MKKRLSSVVIASTVLLGTLTAPMVAMADNYDTQIEQKNSEINDLKAKQSSAQKQIDELEASVAKINKQANELLDQQATLQDESVQLQKDIETLKERIAKREETIQRQARDVQEKNQSSVFIKALLDADSFSDALGRLKAMTTIVNANNDLVNQQKADKKAVEDKKAENEKKQAEIAANQAKLEEQKGTLEAKQADLNVLKSTLAAQQATKESEKEALNAQKAAYEAEQARIRQEQAQVAATRQAVAQQASSSQASASASNTGSSSSSASVSTPAVSIPSTPAPAPSGNGSAIVAEAYKHIGKPYVWGAKGPDTFDCSGFTRYVYLQVTGRDIGGWTVPQESAGTVIPVSQAQPGDLYFWGSRGSTSHVAIAIGGGQYIHAPQPGETVKVGSVAYFAPSFAVRM

SEQ ID NO: 13 - protein, 525 aa, Enterococcus hermanniensis
MKKSLLSALMVCSVTLSALAAPAAASADEFDSKIADQDAKISSLQNQQTDAQSQISALENEVAAINDQAQSLLNNQASLRQKSQDLENQINALQKRIEKRSEAIKEQARNVQVKQSSTNVIDVVLNAESFSDAVGRVQAMSTIVKANNDLVEQQKADKAEVEQKQAENKKQQEAIVANQSALESQKGDLITKQADLNVQTTTLAAEQATAENEKADLQAKQEAAIKEQQRVQEEARQAAAAQEAAQKAEAERQAQADAKAQEEAASSESSTQASTEASTTTVESSTTQESTESSTTTVESSSTEQTAPSTSTTDSSTTTESSTATTEDSSQATEVTPPASSTDTSTSTSNSSNQESSTSTNTSTSTNNSSNQGSSTSTGNSNQGSTSSSNDQTATTPSTPSTSTPAPSGNGAAVVAEAMKYIGTPYVWGGKTPSGFDCSGFTAYVYRQATGREIGGWTVPQESAGTRISVSEAQAGDLYFWGSPGGSYHVAIAMGGGQYIHAPQPGESVKVGSTAYYAPSFAVRM

SEQ ID NO: 14 - protein, 491 aa, Enterococcus devriesei
MKKSLLSALMVCSVTLSALAAPAAASADEFDSKIADQDAKISSLQNQQTDAQSQISALEGEVASINDKAQSLLNEQATLRQKSQDLQKQIKTLQERIEKRSEAIKEQARDVQVKQSSTNVIDVVLNAESFSDAVGRVQAMSTIVKANNDLVEQQKADKVEVEQKQAETQKQQEQITANQAALESQKGDLITKQADLNVQTTTLAAEQATAEGEKANLKDKQAAAIQEQQRVQEEAKKAAEAQEAAQKAEVERQAKADADAKAEADRKAQEEAAASASSTESSTVESSTTEASSTEQTTQSSTVESSTGSTSEDNFQGGGVTPTPTPPSTTPETPTNNENSNSGNQGSNQTPAPTPTPTPTPEPTPAPTPTPAPSGNTAGVVAEAMKYIGTPYVWGGKTPAGFDCSGFTSYVFRQATGREIGGWTVPQESAGTRISVSQAQAGDLYFWGSPGGSYHVAIAMGGGQYIHAPQPGQSVTVSSVSYFAPSFAVRM

SEQ ID NO: 15 - protein, 484 aa, Enterococcus malodoratus
MKKSLLSALMVCSVTLSALAAPAAASADEFDSKIADQDAKISSLENQQTDAQSQINALEGEVASINDKAQGLLNEQASLREKSQELQKQIETLDKRIEKRSETIKEQARDTQVKQSSGSNVIDVVLNAESFTDAVSRVQAMTTIVKANNDLVEQQKADKAEVEQKQAENQKQQEQITANQATLESQKGDLITKQADLNVQTTTLATQQATAESEKASLKEKQDAAVKEQQRVQEEARKAAEAQEAAQKAEADRKAKADSDAKAEADRKAQEEAATSTTTTESSTAESAATVESSSTEQQTQSSATESSSTASTSEDNFQGGGATPTTPSDTGNSSSSDQGSTSSSTNNQTPSTPAPTPTPTPAPAPSGNTGGVVAEAMKYIGTPYVWGGKTPGGFDCSGFTSYVYRQATGREIGGWTVPQESAGTRISVDQAQAGDLYFWGSPGGSYHVAIAMGGGQYIHAPQPGETVTVSSVSYYAPSFAVRM

SEQ ID NO: 16 - protein, 479 aa, Enterococcus avium
MKKSLLSALMVCSVTLSALAAPAAASADEFDSKIADQDAKISSLQNQQTDAQSQISALEGEVASINDKAQGLLNEQESLRQKSQELQKQIETLDKRIEKRSEAIKEQARDTQVKQSSGTNVIDVVLNAESFTDAVSRVQAMTTIVKANNDLVEQQKADKAEVEQKQAENKKQQEQIAANQATLESQKGDLIAKQADLNVQTTTLAAEQATAESDKASLKEKQEAAIKEQQRVQEEARKAAEAQEAAKKADAEAKEKADADAKAEADRKAQEEAAASTTTTESSSSTVESSSTEQTQSSTVDSSATESSNEDNFQGGGATPTTPSESGTGNTNSNNQGSTSSTTNNQTPSTPAPTPTPTPAPSGNGSGVVAEAMKYIGTPYVWGGKTPAGFDCSGFTSYVFRQATGREIGGWTVPQESAGTRISVGEAQAGDLYFWGSPGGTYHVAIAMGGGQYIHAPQPGETVTVSSVSYYAPSFAVRM

SEQ ID NO: 17 - protein, 495 aa, Enterococcus casseliflavus
MKKKIFATVCMCGIVLSSFGGPVTVFATNHDQLIEQKNNEIDQLRQQRQSVQGEIDSLNQEAAIILAQQSDLLQAIEGLDQEISQLEERIAKRSENIEKQARETQVNGKGDNFLTAVLEADSVSDLVGRVHAMTTIIRANNEVIEQQKADQQAVEQKRAESQEKVAELQAAQSHLEAQKGVLEASQAELNVLVSQLAYEEATKEEKEQLRAEKEAYEAEQARIREEAARVAALQAQAEQAAQQQAEQAAAEAAALNEAAVQAEGTEADAEAESPEPAEEPAAPAETQPEETQESEPVETPEAPEEAPVDTPEIQEPETPVTPPAPETPADSAPAVPAPTPAPTPVTPTPAPTPAPSPAPIVTPPAPIVTPPAPSAPASTNGAAIVAEAYKHIGKPYVWGAKGPDSFDCSGFTRYVFLQVTGRDIGGWTVPQETAGTVISVSQAQPGDLLFWGSSGSTYHVAIALGGGQYIHAPRPGQNVSVGSTAHFTPSFAVRM

SEQ ID NO: 18 - protein, 496 aa, Enterococcus gallinarum
MKKKIFATVCMCGIVLSSFGGPVTVFATNHDQLIEQKNNEIDQLRQQRQSVQGEIDGLSAEIDSLNQEAAIILAQQSDLLQAIEGLDQEISQLEERIAKRSENIEKQARETQVNGKGDHFLTAVLEANSVSDLVGRVHAMTTIIRANNEVIEQQKADQQAVEQKRAESQEKVAELQAAQSHLEAQKGVLEASQAELNVLVSNLAYEEATKEEEKEQLRAEKEAYEAEQARIREEAARVAALQAQAEQAAQQQAEQAAAEEAALNEAAVQVESTEPDVETESPAPVEEPEAPAETQPEETQESEPVETPEVPEETPVDTPEIHEPETPVTPATPETPADSAPAVPAPTPAPTPVTPTPAPTPAPSPAPIVTPPAPSAPASTNGAAIVAEAYKHIGKPYVWGAKGPDSFDCSGFTRYVFLQVTGRDIGGWTVPQETAGTVISVSQAQPGDLLFWGSSGSTYHVAIALGGGQYIHAPRPGQNVSVGSTAHFTPSFAVRM

SEQ ID NO: 19 - protein, 92 aa, Enterococcus faecium
GTPYVWGGKDPSGFDCSGFTRYVYLQVTGRDIGGWTVPQESAGTKISVSQAKAGDLLFWGSPGGTYHVAIALGGGQYIHAPQPGESVKVGSV

SEQ ID NO: 20 - protein, 92 aa, Enterococcus villorum
GTPYVWGGKDPSGFDCSGFTRYVYLQVTGRDIGGWTVPQESAGAKISVSQAKAGDLLFWGSPGGTYHVAIALGGGQYIHAPQPGENVKVGSV

SEQ ID NO: 21 - protein, 92 aa, Enterococcus hirae
GTPYVWGGKDPSGFDCSGFTRYVYLQVTGRDIGGWTVPQESAGTKISVSQAKAGDLLFWGSAGGTYHVAISLGGGQYIHAPQPGENVKVGSV

SEQ ID NO: 22 - protein, 92 aa, Enterococcus durans
GVPYVWGGKDPSGFDCSGFTSYVYKQVTGRDIGGWTVPQENAGAKISVSQAKAGDLLFWGSPGGTYHVAIALGGGQYIHAPQPGESVKVGSV

SEQ ID NO: 23 - protein, 92 aa, Enterococcus ratti
GVPYVWGGKDPSGFDCSGFTRYVYLQATGRDIGGWTVPQESAGTRISVSQAKAGDLLFWGSPGGSYHVAISLGGGQYIHAPQPGESVKVGSV

SEQ ID NO: 24 - protein, 92 aa, Enterococcus mundtii
GTPYVWGGKDPSGFDCSGFTRYVYLQVTGRDIGGWTVPQESAGARISVSQAKAGDLLFWGAAGGTYHVAISLGGGQYIHAPQPGESVKIGSV

SEQ ID NO: 25 - protein, 92 aa, Enterococcus hermanniensis
GTPYVWGGKTPSGFDCSGFTAYVYRQATGREIGGWTVPQESAGTRISVSEAQAGDLYFWGSPGGSYHVAIAMGGGQYIHAPQPGESVKVGST

SEQ ID NO: 26 - protein, 92 aa, Enterococcus phoeniculicola
GTPYVWGGKDPSGFDCSGFTSYVYRQATGREIGGWTVPQESAGAKIGINEAQAGDLLFWGSPGGTHHVAIALGGGQYIHAPQPGESVKIGSY

SEQ ID NO: 27 - protein, 92 aa, Enterococcus columbae
GKPYVWGAKGPDTFDCSGFTRYVYLQVTGRDIGGWTVPQESAGTVIPVSQAQPGDLYFWGSRGSTSHVAIAIGGGQYIHAPQPGETVKVGSV

SEQ ID NO: 28 - protein, 92 aa, Enterococcus devriesei
GTPYVWGGKTPAGFDCSGFTSYVFRQATGREIGGWTVPQESAGTRISVSQAQAGDLYFWGSPGGSYHVAIAMGGGQYIHAPQPGQSVTVSSV

SEQ ID NO: 29 - protein, 92 aa, Enterococcus malodoratus
GTPYVWGGKTPGGFDCSGFTSYVYRQATGREIGGWTVPQESAGTRISVDQAQAGDLYFWGSPGGSYHVAIAMGGGQYIHAPQPGETVTVSSV

SEQ ID NO: 30 - protein, 92 aa, Enterococcus raffinosus
GTPYVWGGKTPAGFDCSGFTSYVYRQATGREIGGWTVPQESAGTRISVDQAQAGDLYFWGSPGGSYHVAIAMGGGQYIHAPQPGETVTVSSV

SEQ ID NO: 31 - protein, 92 aa, Enterococcus saccharolyticus
GVPYVWGGKDPSGFDCSGFTSYVYRKATGREIGGWTVPQESAGSVISVSEAKAGDLLFWGSQGSTYHVAIALGGGQYIHAPAPGQSVTVASV

SEQ ID NO: 32 - protein, 92 aa, Enterococcus avium
GTPYVWGGKTPAGFDCSGFTSYVFRQATGREIGGWTVPQESAGTRISVGEAQAGDLYFWGSPGGTYHVAIAMGGGQYIHAPQPGETVTVSSV

SEQ ID NO: 33 - protein, 92 aa, Enterococcus cecorum
GKPYVWGAKGPNTFDCSGFTRYVYLQVTGRDIGGWTVPQEGAGAIIPVSQAQPGDLYFWGSRGSSYHVAIALGGGSYIHAPQPGESVKVGSV

SEQ ID NO: 34 - protein, 92 aa, Enterococcus gilvus; identical to SEQ ID NO: 32

SEQ ID NO: 35 - protein, 92 aa, Enterococcus casseliflavus
GKPYVWGAKGPDSFDCSGFTRYVFLQVTGRDIGGWTVPQETAGTVISVSQAQPGDLLFWGSSGSTYHVAIALGGGQYIHAPRPGQNVSVGST

SEQ ID NO: 36 - protein, 92 aa, Enterococcus gallinarum; identical to SEQ ID NO: 35

(19) United States
(12) Patent Application Publication
(10) Pub. No.: US 2025/0268961 A1
(43) Pub. Date: Aug. 28, 2025

(54) MODIFIED MICROORGANISMS EXPRESSING SagA AND RELATED COMPOSITIONS FOR IMMUNOMODULATION AGAINST INFECTION AND CANCER IMMUNOTHERAPY

(71) Applicant: THE ROCKEFELLER UNIVERSITY, NEW YORK, NY (US)

(72) Inventors: HOWARD HANG, DEL MAR, CA (US); MATTHEW GRIFFIN, NEW YORK, NY (US)

(21) Appl. No.: 19/182,777

(22) Filed: Apr. 18, 2025

Related U.S. Application Data
(62) Division of application No. 17/432,500, filed on Aug. 20, 2021, now Pat. No. 12,280,078.
(60) Provisional application No. 62/807,830, filed on Feb. 20, 2019.

(51) Int. Cl. A61K 35/747; A23C 9/123; A23K 10/18; A23L 33/135; A61K 45/06; A61P 35/00; C07K 14/315

(52) U.S. Cl. CPC A61K 35/747; A23C 9/1234; A23K 10/18; A23L 33/135; A61K 45/06; A61P 35/00; C07K 14/315; A23V 2002/00; A23V 2400/157; A23V 2400/169

(57) ABSTRACT

Provided are compositions and methods based in part on the discovery that Enterococcus faecium secreted antigen A (SagA)-expressing bacteria are protective against enteric infections and enhances immune checkpoint inhibitor efficacy against cancer. Bacteria that express SagA or heterologous SagA, or orthologs thereof, are provided, and are included as nutraceutical, pharmaceutical, and probiotic formulations, as well as components of food products, including dairy products.

### CROSS REFERENCE TO RELATED APPLICATIONS

This application is a divisional of U.S. application Ser. No. 17/432,500, filed Aug. 20, 2021, now U.S. Pat. No. 12,280,078, which is the U.S. National Stage Application of International Patent Application No. PCT/US2020/019038, filed Feb. 20, 2020, which claims the benefit of U.S. Provisional Application Ser. No. 62/807,830, filed Feb. 20, 2019, the disclosures of which are hereby incorporated by reference in their entirety, including all figures, tables and amino acid or nucleic acid sequences.

### STATEMENT REGARDING FEDERALLY SPONSORED RESEARCH

This invention was made with government support under contract no. R01 GM103593 awarded by the National Institutes of Health. The government has certain rights in the invention.

### STATEMENT REGARDING SEQUENCE LISTING

The Sequence Listing for this application is labeled “Seq-List.xml” which was created on Apr. 4, 2025 and is 42,377 bytes. The entire content of the sequence listing is incorporated herein by reference in its entirety.

### FIELD

The present disclosure relates generally to use of naturally occurring and recombinant microorganisms expressing secreted antigen A (SagA) proteins as anti-infective agents, as well as for use in stimulating immune responses and potentiating vaccine and anti-cancer agents.

### BACKGROUND

Cancer remains a significant health burden and is the second leading cause of mortality in the U.S. and worldwide, with an estimated 9.6 million deaths globally in 2018. However, the disease results from a myriad of different causes with quite variable genotypes and drug susceptibilities, requiring the development of many targeted therapies and individualized treatment regimens. Fortunately, cancer immunotherapies such as immune checkpoint inhibitors (ICIs) have exhibited broad success in the clinic against diverse cancer types. For example, six separate antibodies targeting PD-1/PD-L1 have been approved by the FDA to treat melanoma, Hodgkin's lymphoma, renal cell carcinoma, hepatocellular carcinoma, cervical, non-small-cell lung, colorectal, gastric, kidney, bladder, head and neck cancers and others. Moreover, anti-PD-1/PD-L1 drugs are currently being tested in single or combination therapy in over 2,250 active clinical trials as of September 2018, underscoring both the great potential and wide impact of these novel immunotherapies. Nevertheless, not all patients respond to ICIs, and the underlying determinants that distinguish non-responding versus responding patients remain unclear. Thus, identifying mechanisms that alter the efficacy of ICI therapies is an ongoing need. In this regard, commensal microbes within the human gut play a critical role in the efficacy of immunotherapies and other cancer drugs, including anti-CTLA4 and anti-PD-1/PD-L1, indicating that the gut microbiota is a necessary component of immunotherapeutic modes of action. Although these and other studies correlate the microbiota composition and cancer immunotherapy response, the causative factors from the microbiota that lead to improved drug efficacy are unknown. Thus, there is an ongoing and unmet need to provide new and improved compositions and methods that can harness the benefit of microbiota on immunotherapy and other outcomes. The present disclosure is pertinent to this need.

### BRIEF DESCRIPTION OF THE DRAWINGS

FIG. 1. Representative orthologs of Enterococcus faecium secreted antigen A (SagA).

FIG. 2. Full sequences of representative SagA orthologs.

FIG. 3. C-terminal NlpC/p60 domains of SagA orthologs (Inter-Pro) and Percent ID (MView, EMBL-EBI).

FIGS. 4A-4B. Tumor growth model in C57BL/6 mice to evaluate Enterococci strains during PD-L1 therapy. FIG. 4A) Abx-pretreated specific-pathogen-free (SPF) mice are colonized with different Enterococcus strains, inoculated with B16/F10 cells and evaluated for tumor growth, alterations in immune responses and microbiota composition. FIG. 4B) Tumor growth of 1×10⁵ B16/F10 cells injected into C57BL/6 mice with or without anti-PD-L1 treatment and Enterococci colonization (n=8 per condition, mean±s.e.m.).

FIGS. 5A-5B. SagA sequence identity, SagA expression and peptidoglycan profile of Enterococci species. FIG. 5A) Schematic of SagA-like proteins from Efm, Edr and Ehr with percent identity compared to Efm SagA domains. FIG. 5B) Western blot of SagA from different Enterococcus species.

FIGS. 6A-6C. Activity of Lpl-sagA strains against Cdf infection in mice. FIG. 6A) Schematic summary of wild-type and mutant SagA-His6 constructs expressed in Lpl. FIG. 6B) Expression and secretion levels of wild-type and mutant SagA-His6 constructs expressed in Lpl. FIG. 6C) Survival curve of C. difficile infected mice. Mice were treated for 7 days with Abx cocktail and then orally gavaged with PBS or 5×10⁸ CFU of L. plantarum (Lpl) containing empty vector or a vector expressing a SagA variant followed by Cdf infection. Pooled data from 3 independent experiments, n=9-10 mice/group.

FIGS. 7A-7B. Tumor growth model in C57BL/6 mice to evaluate Enterococci strains naturally or recombinantly expressing SagA during anti-CTLA-4 or anti-PD-1 immunotherapy. FIG. 7A) Tumor growth of 3×10⁵ MC-38 cells injected into C57BL/6 mice with or without anti-CTLA-4 treatment and Enterococci colonization (n=8 per condition, mean±s.e.m.). FIG. 7B) Tumor growth of 6×10⁵ MCA205 cells injected into C57BL/6 mice with or without anti-PD-1 treatment and Enterococci colonization (n=8 per condition, mean±s.e.m.).

FIGS. 8A-8C. SagA sequence identity, SagA expression and tumor growth model in C57BL/6 mice to evaluate different Enterococci strains. FIG. 8A) Western blot of SagA from different Enterococcus strains. FIG. 8B) Colonization of gastrointestinal tract by different Enterococcus species. FIG. 8C) Tumor growth of 1×10⁵ B16/F10 cells injected into C57BL/6 mice with anti-PD-1 treatment and/or Enterococci colonization of different Enterococci spp. (n=8 per condition, mean±s.e.m.).

FIGS. 9A-9B. Tumor growth model in SPF-Taconic mice to evaluate Enterococci strains during ICI therapy in an organism with a non-responsive microbiota. FIG. 9A) Colonization of gastrointestinal tract by different Enterococcus species. FIG. 9B) Tumor growth of 1×10⁵ B16/F10 cells injected into SPF-Taconic mice with anti-PD-L1 treatment and Enterococci colonization (n=8 per condition, mean±s.e.m.).

FIGS. 10A-10B. Tumor growth model in NOD2^{-/-} and NOD2^{+/-} mice to evaluate the role of Nod2 during ICI therapy. FIG. 10A) Individual tumor growth of 1×10⁵ B16/F10 cells injected into NOD2^{-/-} and NOD2^{+/-} mice with anti-PD-L1 treatment and Enterococci colonization (n=8 per condition). FIG. 10B) Mean tumor growth of 1×10⁵ B16/F10 cells injected into NOD2^{-/-} and NOD2^{+/-} mice with anti-PD-L1 treatment and Enterococci colonization (n=8 per condition, mean±s.e.m.).

FIG. 11. SagA-L. lactis restores immune checkpoint inhibitor efficacy. L. lactis (Lls) lines express either wild-type SagA (Lls WT), catalytically inactive SagA (Lls CA), or SagA variant that does not contain the signal sequence and is not secreted (Lls delSS). Mean tumor growth of 1×10⁵ B16/F10 cells injected into C57BL/6 mice with anti-PD-L1 treatment and Lls, Lls WT, Lls CA, Lls delSS, or Efm dosing (n=8 per condition).

FIGS. 12A-12B. L. lactis expressing heterologous SagA protects against survival in a murine C. difficile infection model. To establish the C. difficile infection model, mice were gavaged with the AMNV (4 mg ampicillin, 2 mg metronidazole, 4 mg neomycin, 2 mg vancomycin) antibiotic cocktail daily for 7 days before receiving an oral administration of clindamycin (10 mg/kg). Two days later mice (8/group) were treated by oral gavage with phosphate-buffered saline (PBS), 1×10⁹ CFU of negative control L. lactis MG1363 (Lactis Control), or 1×10⁹ CFU of L. lactis expressing SagA (Lactis SagA). Mice were then infected with C. difficile 30 hours after treatment. FIG. 12A illustrates the number of surviving mice and FIG. 12B illustrates the weight of the mouse each day, unless the mouse was euthanized before the 10 days expired.

### BRIEF DESCRIPTION OF SEQUENCES

SEQ ID NO: 1 is the protein sequence of the heterologous SagA.

SEQ ID NOs: 2-18 are sequences of representative SagA orthologs.

SEQ ID NOs: 19-36 are sequences of C-terminal NlpC/p60 domains of SagA orthologs (Inter-Pro) and Percent ID (MView, EMBL-EBI).

### DETAILED DESCRIPTION OF THE INVENTION

As used herein, the singular forms “a”, “an” and “the” are intended to include the plural forms as well, unless the context clearly indicates otherwise. Furthermore, to the extent that the terms “containing”, “including”, “includes”, “having”, “has”, “with”, or grammatical variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” or “approximately” means within an acceptable error range for the particular value as determined by one of ordinary skill in the art, which will depend in part on how the value is measured or determined, i.e., the limitations of the measurement system. The terms “about” and “approximately” are meant to encompass a range of ±20%, ±10% or ±5% of a given value. Thus, in the context of compositions containing amounts of ingredients where the terms “about” or “approximately” are used, these compositions can contain the stated amount of the ingredient with a variation (error range) of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The terms “simultaneous” or “simultaneously” as applied to the method of treating an individual with cancer using SagA refers to adding one or more components to the subject at the same time, or at two different time points that are separated by no more than 3 minutes. The phrase “after or before” as applied to methods of treating an individual with cancer refers to providing more than one composition at two different time points that are separated by more than 3 minutes, e.g., about 5 minutes, 30 minutes, 1 hour, about 2 hours, about 5 hours, or even longer.

The present disclosure is based in part on our discovery that Enterococcus faecium can activate host immunity and inhibit pathogenesis by various strains of bacteria, and also function to enhance certain anti-cancer approaches.

The disclosure includes all amino acid sequences described herein, and all polynucleotides encoding the amino acid sequence. The disclosure also includes all amino acid sequences that have at least 80% similarity to any amino acid sequence described herein. The percent amino acid sequence can be determined across the full length of any amino acid sequence described herein, or across any contiguous amino acid sequence that constitutes a functional domain, such as a NlpC/p60 domain, non-limiting examples of which are provided below. As such, the disclosure includes sequences that are the same as any amino acid sequence described herein, and from 80, 81, 82, 83, 84, 85, 86, 87, 88, 89, 90, 91, 92, 93, 94, 95, 96, 97, 98, 99% identical to such sequences. Likewise, the disclosure includes each type of bacteria described herein, and all combinations of such bacteria, and compositions comprising said bacteria and bacteria combinations, and methods comprising administering such bacteria and bacteria combinations to an individual, with or without vaccine or immune checkpoint inhibitor, as further described below. In one aspect the present disclosure provides a modified microorganism, such as modified bacteria, wherein the organism expresses a heterologous SagA. A “heterologous” SagA protein is a SagA protein that is not normally encoded by the genome of the microorganism. Accordingly, heterologous SagA production involves introducing a SagA-encoding DNA sequence into the microorganism. The modified microorganism comprises a heterologous SagA coding sequence, the expression of which is driven by a promoter operative in the microorganism. The SagA protein can be expressed from any suitable expression vector or other construct introduced into the microorganism. In embodiments the heterologous SagA is encoded by a plasmid introduced into the modified microorganism, or is encoded by a segment of DNA introduced into a bacterial chromosome. In embodiments, the SagA encoded by modified bacteria is a truncated and/or mutated SagA, which may be referred to as a SagA component.

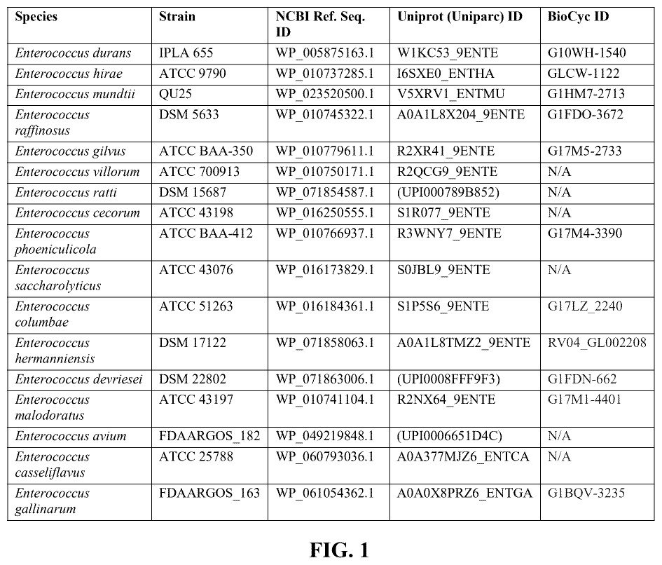

Many reagents and methods for introducing and expressing any heterologous gene in a wide variety of microorganisms are known in the art and are suitable for use with the present invention. In general, the disclosure contemplates microorganisms that are modified to express and secrete heterologous SagA that retains the ability to generate peptidoglycan fragments from a suitable peptidoglycan-containing substrate. In one embodiment, the recombinantly produced SagA protein comprises the amino acid sequence:
MKKSLISAVMVCSMTLTAVASPIAAAADDFDSQIQQQDQKIADLKNQQADAQS QIDALESQVSEINTQAQDLLAKQDTLRQESAQLVKDIADLQERIEKREDTIQKQAREAQ VSNTSSNYIDAVLNADSLADAIGRVQAMTTMVKANNDLMEQQKQDKKAVEDKKAEN DAKLKELAENQAALESQKGDLLSKQADLNVLKTSLAAEQATAEDKKADLNRQKAEAE AEQARIREQQRLAEQARQQAAQEKAEKEAREQAEAEAQATQASSTAQSSASEESSAAQ SSTTEESSSAAQSSTTEESTTAPESSTTEESTTAPESSTTEESTTVPESSTTEESTTVPESSTT EESTTVPESSTTEESTTVPETSTEESTTPAPTTPSTDQSVDPGNSTGSNATNNTTNTTPTPT PSGSVNGAAIVAEAYKYIGTPYVWGGKDPSGFDCSGFTRYVYLQVTGRDIGGWTVPQE SAGTKISVSQAKAGDLLFWGSPGGTYHVAIALGGGQYIHAPQPGESVKVGSVQWFAPD FAVSM (SEQ ID NO: 1), or a sequence that has at least 80% identity to this sequence, provided such non-identical sequences retain NlpC/p60-type hydrolase activity. In this representative sequence, the NlpC/p60 hydrolase domain is from amino acid 389 through amino acid 530. In embodiments, other E. faecium SagA sequences are known and would be expected to function in place of Com15 SagA, the sequence of which is given above. In embodiments, the SagA protein is from any of the following types of bacteria, and the disclosure includes any amino acid sequence that has at least 80% identity to such SagA sequences, provided non-identical sequences retain NlpC/p60-type hydrolase activity that are listed in FIGS. 1, 2 and 3. The polynucleotide and amino acid sequences from each database entry stated in this disclosure are incorporated herein as they exist on the filing date of this application or patent.

In embodiments, the SagA protein is modified so that it has, for example, additional or fewer amino acids than in the sequence presented above. In non-limiting examples, the SagA protein is modified to include additional amino acids used for isolation, purification, or detection, including but not necessarily limited to amino acid residues in the C-terminus, or a polypeptide sequence that is capable of producing a detectable signal, such as a fluorescent signal.

In embodiments, the disclosure includes modified bacteria that express heterologous SagA protein, with the proviso that the gram-negative bacteria do not include Escherichia coli. In embodiments, the disclosure includes modified bacteria that are facultative anaerobes. In embodiments the modified bacteria are gram-positive and gram-negative bacteria that express heterologous SagA protein. In embodiments the gram-positive bacteria are members the Lactobacillus genus, and in particular Lactobacillus species that are active in the production of food products intended for human and/or non-human animal consumption. In non-limiting embodiments the modified bacteria are Lactobacillus species that are active in the production of dairy products, such as yogurt, milk, milk-based creams, ice cream products, and cheese, or fermented drinks, such as wine, cider and beer, or fermented foods, or combinations of the foregoing. In certain embodiments the modified bacteria are L. plantarum, L. casei, L. acidophilus, L. salivarius, or L. reuteri as well as probiotic strains of Lactococcus lactis and Bifidobacterium (e.g., B. longum).

In embodiments, the disclosure includes combinations of modified bacteria described herein, and further comprises combinations of the modified bacteria with other microorganisms, such as yeasts. Those skilled in the art will recognize that such combinations are useful for production of certain foods.

In another aspect, the disclosure comprises a food product comprising modified bacteria that expresses a heterologous SagA protein. Such products include all of the aforementioned types of food and modified bacteria, and may further include modified bacteria that express a heterologous SagA. In embodiments the food product is a dairy product, including but not necessarily limited to yogurt, milk, milk-based creams, and cheese. Use of microorganisms in making foods that intentionally contain live cultures, such as yogurts, are well known in the art and can be adapted for use with the presently provided modified microorganisms. In one aspect the food product is a non-human animal feed, such as food intended for consumption by a bovine, equine, canine, porcine, feline, avian or reptilian animal, or by aquatic animals such as fish. In certain aspects the food product comprises packaging, such as a paper or cardboard carton, plastic container, bottle, bag, etc., that are well known for containing foods. The packaging can provide printed material which includes information that identifies the modified bacteria present in the food product.

In another aspect, the disclosure includes a supplement product, such as a nutraceutical product, a dietary supplement, a food ingredient, etc., including but not limited to a probiotic formulation or functional food that contains one or more live modified bacteria as described herein. The supplement product can be provided in the form of, for example, a liquid, capsules, tablets, soft gels, powders, freeze-dried compositions, and the like.

In another aspect, the disclosure provides a pharmaceutical composition comprising modified microorganisms and/or isolated or purified recombinant SagA as described herein. The pharmaceutical composition can include any suitable diluent, carrier, excipient, buffer, etc., intended for use with the microorganisms for prophylactic and/or therapeutic human or veterinary purposes. Some examples of compositions suitable for preparing pharmaceutical compositions can be found in: Remington: The Science and Practice of Pharmacy (2005) 21st Edition, Philadelphia, PA. Lippincott Williams & Wilkins. Such compositions may also be included in supplement products.

In an embodiment the disclosure includes making modified bacteria that express heterologous SagA for use in inhibiting bacterial infections, or for maintaining or modifying the intestinal flora of an individual. The method comprises introducing into bacteria a heterologous SagA encoding DNA sequence, and culturing the bacteria for use as a probiotic, nutraceutical or pharmaceutical agent. In embodiments, the disclosure comprises such bacterial cultures themselves, and further includes such cultures scaled for use in producing nutraceutical, probiotic and/or pharmaceutical preparations. In embodiments, the cultures are propagated as, for example, a yogurt culture. The disclosure also includes composition and methods that employ bacteria that are unmodified, at least with respect to their expression of SagA or a SagA ortholog, non-limiting examples of such SagA orthologs being presented in FIGS. 1-3. In embodiments, the bacteria express a SagA ortholog that comprises an amino acid sequence that is at least 80% identical to the SagA-NlpC/p60 hydrolase domain, non-limiting examples of which are shown at least in FIG. 3. In embodiments, a composition comprising only a single type or strain of bacteria is provided and/or administered to an individual. In embodiments, a mixture of distinct types/strains of bacteria is provided and/or is administered to an individual.

In another aspect, the disclosure includes a method comprising introducing into modified bacteria, wherein the modified bacteria expresses a heterologous SagA. The modified bacteria can be introduced as a component of a food product, a probiotic formulation, or a pharmaceutical formulation. In an aspect the disclosure includes a method for prophylaxis and/or therapy of a bacterial infection in an individual. The method comprises administering to an individual in need a composition comprising isolated SagA, or a bacterial population wherein at least some members of the population have been modified to produce SagA. In another approach, the composition can comprise peptidoglycan fragments generated by SagA. Compositions and uses thereof comprising combinations of peptidoglycan fragments generated by SagA, modified bacteria that express SagA and isolated SagA are also encompassed by this disclosure. Compositions of the disclosure may be used prophylactically when they are given to an individual prior to exposure to pathologic bacteria, or within a short time, i.e., several hours, after exposure to pathologic bacteria. Therapeutic approaches comprise administering the engineered bacteria to an individual who has or is suspected of having a bacterial infection, wherein the severity of the infection is lessened subsequent to the administration.

In embodiments, administering a composition to an individual for, for instance, prophylaxis and/or therapy of a bacterial infection according to this disclosure can be performed using any suitable approach. In one embodiment, the composition is consumed by the individual as a probiotic formulation, or as a component of a food item and is thus introduced orally. In another embodiment the composition is administered as a pharmaceutical formulation. The pharmaceutical formulation can be administered using any suitable route, including but not necessarily limited to parenteral, intraperitoneal, intrapulmonary, oral, intra-abdominal, and others. Parenteral infusions include intramuscular, intravenous, intraarterial, intraperitoneal, and subcutaneous administration.

The amount of modified bacteria, SagA, and any other active agent to be included in a composition and/or to be used in the method can be determined by those skilled in the art, given the benefit of the present disclosure. Thus, in one embodiment, an effective amount of a composition is administered. An effective amount can be an amount of the composition that inhibits growth of bacterial cells in the individual, or reduces a sign or symptom of bacterial infection. In embodiments, the individual to whom a composition of the invention is administered has, is suspected of having, or is at risk for development of a bacterial infection. In embodiments, the bacterial infection is an infection by enteric pathogens such as strains of Salmonella, Escherichia coli, Shigella, Campylobacter, Helicobacter, Francisella, Vibrio, Yersinia, Enterococcus, Clostridia and pathogenic strains of microbiota. In embodiments, a composition of this disclosure is administered to an individual such that the growth of enteric pathogens in the individual is inhibited, and/or the amount of enteric pathogens is reduced, or the enteric pathogens are eradicated.

Suitable dosages for either therapeutic or prophylactic purposes can be determined by those skilled in the art and will be based, at least in part, on consideration of the individual's age, sex, size, and health, the type of bacterial infection, and other factors as will be apparent to the skilled artisan. In embodiments, a composition of the invention can be administered in combination with an antibiotic.

In one aspect, the disclosure relates to methods for cancer therapy comprising administering to an individual in need a combination of an immune checkpoint inhibitor (ICI) and one or more SagA proteins, fragments thereof, and/or one or more types of bacteria that express SagA or a derivative thereof or bacteria that express heterologous SagA (collectively referred to herein as “SagA components”).

In embodiments, the individual in need of treatment in accordance with this disclosure is any mammal, including but not limited to a human. The cancer type is not particularly limited, other than being a cancer type for which immune checkpoint inhibition may be a suitable prophylactic and/or therapeutic approach. In embodiments, the individual is at risk for, is suspected of having, or has been diagnosed with a cancer. In embodiments, the cancer is melanoma, lung, colon, breast, pancreatic, brain, liver, bladder, kidney, melanoma, ovary, testicular, esophageal, gastric, fibrosarcoma, rhabdomyosarcoma, head and neck, renal cell, thyroid, or a blood cancer.

The disclosure is also pertinent to approaching cancers that are or may become resistant to treatment with one or more immune checkpoint inhibitors. Thus, in certain implementations the individual has been previously treated for cancer with a checkpoint inhibitor but was not treated with a SagA component while being treated with the checkpoint inhibitor; and, the cancer was initially resistant, or develops resistance, to the checkpoint inhibitor treatment. The disclosure thus includes selecting an individual who has cancer that is resistant to a checkpoint inhibitor as a monotherapy, and administering to the individual a checkpoint inhibitor and a SagA component, as described herein. The individual who is resistant to a checkpoint inhibitor as a monotherapy accordingly means an individual who was administered a checkpoint inhibitor for the cancer but was not also administered a SagA component; and, the cancer was resistant to the treatment that included the checkpoint inhibitor but not the SagA component. The monotherapy may have included other anti-cancer agents or other interventions, so long as such other agents did not include a SagA component that is subsequently used in a combination therapy of this disclosure. In one embodiment the individual who is treated with a combination approach described herein has never been previously treated with a SagA component. In certain embodiments, the individual who is treated with a combination therapy of this disclosure has not been diagnosed with, is not suspected of having, or is not a risk for developing a non-cancerous condition for which a SagA component would be prescribed.

In certain embodiments, a combination of an immune checkpoint inhibitor and a SagA component exerts a synergistic effect against cancer, which may comprise but is not limited to a greater than additive inhibition of cancer progression, and/or a greater than additive inhibition of an increase in tumor volume, and/or a reduction in tumor volume, and/or a reduction in tumor growth rate, and/or an eradication of a tumor and/or cancer cells. The method may also result in a prolonging of the survival of the individual.

The disclosure also comprises monitoring the treatment of an individual who is receiving a combination of an immune checkpoint inhibitor and a SagA component. This approach comprises administering the combination of an immune checkpoint inhibitor and a SagA component as a cancer treatment, testing the individual and/or a biological sample from the individual to determine the efficacy of the combination therapy, and if determined to be necessary, adjusting the combination therapy by, for example, changing the amount of the immune checkpoint inhibitor or the a SagA component, or both, and/or changing the type of immune checkpoint inhibitor and or the a SagA component. Retesting and changing the combination therapy may also be performed.

The immune checkpoint inhibitor used in combination with the one or more SagA components described herein can be any immune checkpoint inhibitor. As is known in the art, an example of an immune checkpoint is the transmembrane programmed cell death 1 protein (PDCD1, PD-1; also known as CD279) and its ligand, PD-1 ligand 1 (PD-L1, CD274). In normal, non-malignant physiology, PD-L1 on the surface of a cell binds to PD-1 on the surface of an immune cell, which inhibits the activity of the immune cell. PD-L1 up-regulation on cancer cell surfaces is thought to facilitate evasion of the host immune system, at least in part by inhibiting T cells that would otherwise target the tumor cell. In alternative embodiments, other immune checkpoints can be inhibited, such as CTLA-4.

In embodiments, any one or more checkpoint inhibitors can be combined with any one or more SagA components described herein for use in the methods of this disclosure. In certain embodiments, the checkpoint inhibitors that are combined with the SagA component comprise antibodies that bind to PD-1, or anti-PD-L1, such as nivolumab, pembrolizumab, durvalumab, atezolizumab, and avelumab. In another embodiment, the checkpoint inhibitor is an antibody that targets CTLA-4, such as ipilimumab and tremelimumab. In another embodiment the checkpoint inhibitor is targets CD366 (Tim-3), which is a transmembrane protein also known as T cell immunoglobulin and mucin domain containing protein-3.

In alternative embodiments, the checkpoint inhibitors comprise small molecules or other agents that disrupt the immune checkpoint that is exploited by cancer cells to evade cell-mediated or other immune-mediated targeting.

Those skilled in the art, given the benefit of the present disclosure, will recognize how to determine an effective amount of the combination of checkpoint inhibitor and a SagA component for treatment of cancer. In general, and without intending to be bound by any particular theory, it is expected that the amounts of each checkpoint inhibitors that are used and/or tested currently in humans for their separate indications will also be effective in the presently provided combination approach. But modifications can be made by medical professionals based on known conditions, such as the size, age, gender and overall health profile of the individual, the type and stage of the cancer, and other conditions and risk factors that will be otherwise apparent to those skilled in the art. In embodiments, administering the checkpoint inhibitor and a SagA component has a greater than additive effect on tumor inhibition, relative to use of either agent alone. A greater than additive effect can be determined by comparing the effects of one or both of the agents to any suitable reference, including but not limited to a predetermined value.

In embodiments, one or more SagA components and one or more immune checkpoint inhibitors are administered concurrently. In embodiments, the one or more SagA components and one or more immune checkpoint inhibitors are combined into a single pharmaceutical formulation. In embodiments, the one or more SagA components and the one or more immune checkpoint inhibitors are administered sequentially. The SagA components and immune checkpoint inhibitor can be administered via any suitable route, including but not necessarily limited to intravenous, intramuscular, subcutaneous, oral, and parenteral routes.

In an embodiment, the combination therapy has a greater than additive inhibition of tumor growth, which may be determined using any suitable measurement, non-limiting examples of which include determining tumor volume or tumor growth rate. The combination therapy can be combined with any other, conventional cancer therapies, including but not limited to surgical and chemotherapeutic approaches.

In alternative embodiments, one or more SagA components described herein are administered in combination with immunotherapy regimens, such as T-cell transfer therapy, antibodies targeting different cancers, a cancer vaccine, and/or immune system modulators. Methods of T-cell transfer therapy are well-known in the art, including tumor infiltrating lymphocytes therapy and CAR T-cell therapy. Antibodies, such as the antibodies available on the IMGT monoclonal antibody database website (see Worldwide Website: imgt.org/mAb-DB), can be administered to a patient in need of treatment of cancer. A cancer vaccine can also be used to treat cancer in an individual. In addition to administering antibodies to immune checkpoint inhibitors, other immune system modulators can be used including cytokines, such as interferons and interleukins, or immunomodulatory drugs, such as thalidomide, lenalidomide, pomalidomide, or imiquimod. The following description and specific Example is provided to illustrate the invention, but are not intended to be limiting in any way.

### EXAMPLES

A greater understanding of the present invention and of its many advantages may be had from the following examples, given by way of illustration. The following examples are illustrative of some of the methods, applications, embodiments, and variants of the present invention. The examples are not to be considered as limiting the invention.

### Example 1—SagA is Sufficient to Improve Efficacy of Immune Checkpoint Inhibitor Antibodies

This example pertains to specific microbial and host factors involved in Enterococci-mediated improvement of ICI therapy to exploit host-microbiota interactions during cancer immunotherapy and develop new ICI therapeutic approaches.

In particular, and as alluded to above, based on our data in mammalian cells and mouse models, embodiments of this disclosure include use of Enterococcus faecium (Efm) and other SagA-expressing Enterococcus species to improve cancer immunotherapy efficacy through their unique peptidoglycan composition and remodeling activity. In this regard, Enterococci and immunotherapy efficacy remains mostly correlative, and individual species have not been directly evaluated during cancer immunotherapy treatment.

In embodiments, these effects are tested using in vivo tumor models, immune profiling and microbiota analysis upon colonization with individual Enterococcus species to provide new protective factors/pathways for cancer treatment. For example, the analysis of Enterococci SagA provides a basis for development of engineered probiotics to enhance cancer immunotherapy. In connection with this, it has already been demonstrated that SagA can be expressed in the probiotic species Lactobacillus plantarum (Lpl) and confer protective activity against intestinal pathogens (see, for example, PCT publication WO 2016/172476), highlighting how beneficial commensal bacteria factors may be successfully transferred into existing probiotic strains.

In non-limiting embodiments, the disclosure includes assessing, using non-limiting examples of SagA-expressing Enterococci, to assess whether colonization is sufficient to improve anti-PD-L1, anti-CTLA-4, or anti-PD-1 efficacy. In embodiments, the well-established tumor growth model with B16/F10 syngeneic melanoma cells, MC-38 adenocarcinoma cells, or MCA205 fibrocarcinoma cells in C57BL/6 mice is used to illustrate certain approaches of this disclosure. For this approach, in a non-limiting example, six-to ten-week-old male and female mice are first treated with an antibiotic (Abx) cocktail (1 g/L ampicillin, 1 g/L colistin sulfate and 5 g/L streptomycin) for 14 days to deplete the endogenous microbiota and facilitate colonization by Enterococcus species.

On the day prior to administration, bacteria were inoculated into 4 mL of autoclaved growth medium and grown as overnight cultures. On the following day, overnight cultures were used to inoculate 50 mL of growth medium at a dilution ratio of 1:50. Bacteria were grown to late logarithmic phase (OD ˜1), centrifuged at 5,000×g for 10 min, and then resuspended in sterile-filtered drinking water. Bacteria were then diluted in two 50-mL aliquots per animal cage in sterile conical tubes to 10⁸ CFU/mL as previously determined by dilution plating. Tubes were then fitted with autoclaved #6 sipper tubes and provided to the animals ad libitum. For Enterococcus strains and species, the bacterial solutions were replaced at least twice weekly. For Lactococcus lactis strains, solutions were replaced every other day. Supplemented drinking water was maintained throughout the remainder of the experiment. As controls, separate cohorts are inoculated with the non-protective species Enterococcus faecalis (Efs, strain OG1RF) or vehicle only.

After 24 h, the animals are then subcutaneously injected with either B16/F10 melanoma cells, MC-38 adenocarcinoma cells, or MCA205 fibrocarcinoma cells. B16/F10 and MCA205cells were cultured at 37° C. and 5% CO₂ in complete DMEM (ThermoFisher, 11995065) supplemented with 10% fetal bovine serum, 100 μg/mL penicillin, and 100 μg/mL streptomycin. MC-38 cells were cultured at 37° C. and 5% CO₂ in the medium described above supplemented with 0.1 mM non-essential amino acids. The final amount of injected cells for each cell type is as follows: B16/F10—1×10⁵ cells; MC-38—3×10⁵ cells; MCA205—6×10⁵ cells.

Tumor growth is quantified by digital calipers two to three times per week for a period of time, such as at least 2.5 weeks.

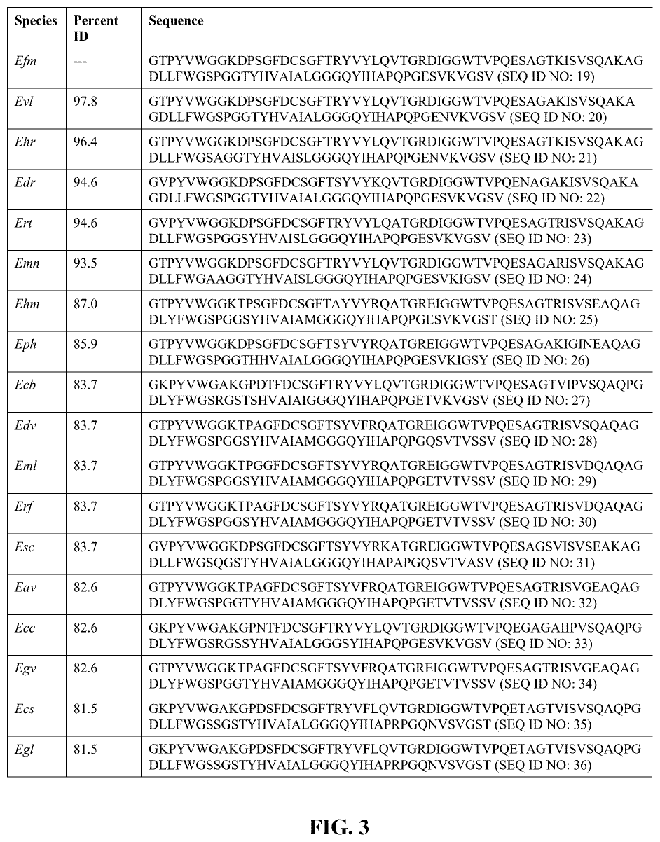

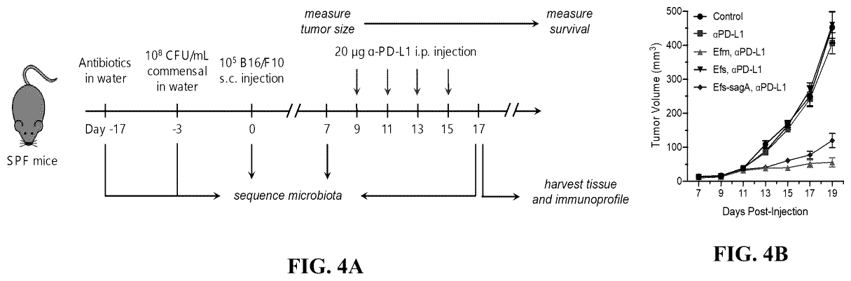

Starting on day 9 post-injection, mice are intraperitoneally (i.p.) injected with antibodies, such as every two days for four total injections. The antibodies and amount per injection used are as follows: 20 μg anti-PD-L1 (BioXCell, clone 10F.9G2—20 or 100 μg; anti-PD-1 (BioXCell, BP0146)—100 μg; anti-CTLA-4 (BioXCell, BP0131)—100 μg. As a negative control, one of the vehicle-inoculated cohorts is i.p. injected with the antibody dilution buffer. Survival may be measured, with endpoints defined as the first of >20% weight loss, 1 cm³ tumor volume, tumor necrosis, or visual signs of distress or pain. All tumor growth experiments can be statistically analyzed by linear mixed modeling. Survival is estimated by Kaplan-Meier, and survival curves are analyzed by log rank test corrected for multiple comparisons (FIG. 4A).

Experiments indicate that Efm and Efs-sagA, but not Efs can inhibit tumor growth after anti-PD-L1 treatment (FIG. 4B). Data presented herein indicate that heterologous expression of SagA in otherwise inactive bacteria (Efs) can significantly improve anti-PD-L1 therapy in a murine model of melanoma (FIG. 4B).

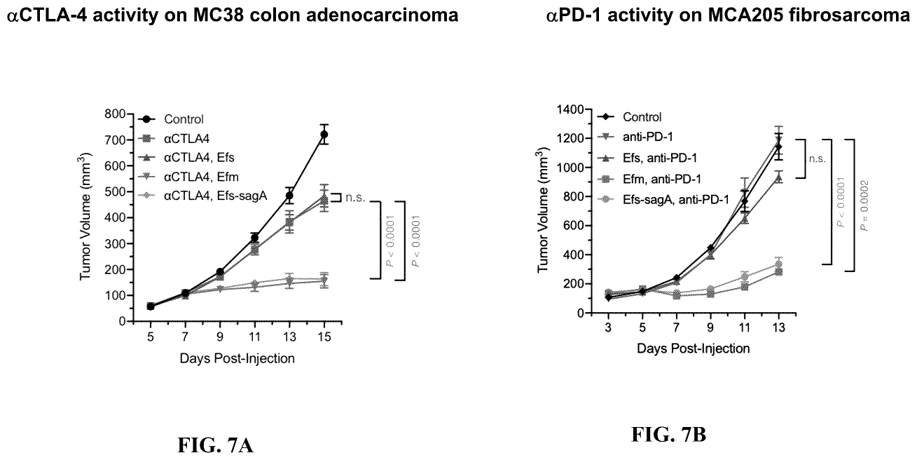

Experiments indicated that Efm and Efs-sagA, but not Efs can inhibit tumor growth after anti-CTLA-4 treatment in a murine model of colon adenocarcinoma (FIG. 7A). Similarly, experiments indicated that Efm and Efs-sagA, but not Efs can inhibit tumor growth after anti-PD-1 treatment in a murine model of fibrosarcoma (FIG. 7B). The data presented indicate that heterologous expression of SagA can significantly improve anti-CTLA-4 and anti-PD-1 therapies in murine models.

### Example 2—SagA Orthologs are Present Throughout the Enterococcus Genus

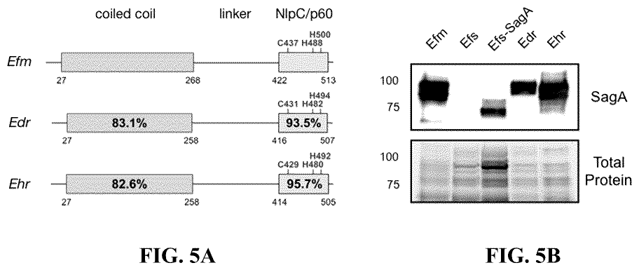

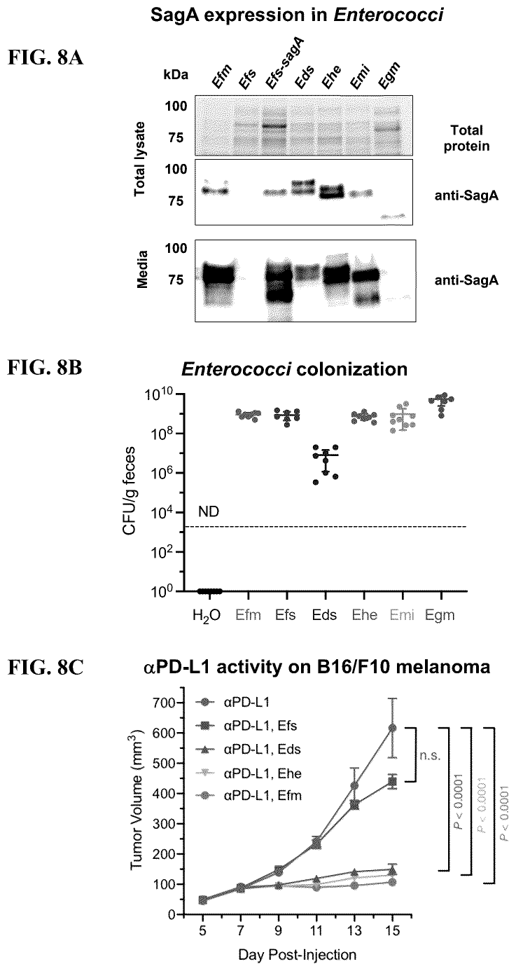

Comparative genomic analysis between other Enterococci species and Efs suggests that immunotherapy-associated Enterococci species express unique bacterial adhesion, metabolism and peptidoglycan remodeling. Remarkably, amongst these uniquely expressed genes, our previously characterized peptidoglycan hydrolase SagA is highly conserved across these strains at the protein level (FIG. 5A). More detailed sequence analysis shows that Edr and Ehr contained SagA-like orthologs with high conservation in the C-terminal NlpC/p60 catalytic hydrolase domain of Efm SagA (FIG. 5A). To confirm the expression of SagA orthologs in Edr, Eds, Ehe, Emi, Egm, and Ehr, overnight cultures of each strain were separately grown along with Efm, Efs-sagA and Efs as positive and negative controls, respectively. Western blotting with a polyclonal antibody against Efm SagA recently generated in our lab showed robust signal in Edr and Ehr (FIG. 5B) and in Efm, Eds, and Ehe (FIG. 8A), demonstrating that Efm SagA-like proteins are produced and secreted in other Enterococcus strains. Our studies show that Efm and Efs-sagA, but not Efs, can enhance anti-PD-L1, anti-CTLA-4, and anti-PD-1 activities against tumor growth (FIG. 4B, FIG. 7A, and FIG. 7B, respectively). Additionally, Eds and Ehe can enhance anti-PD-L1 activity against tumor growth (FIG. 8C).

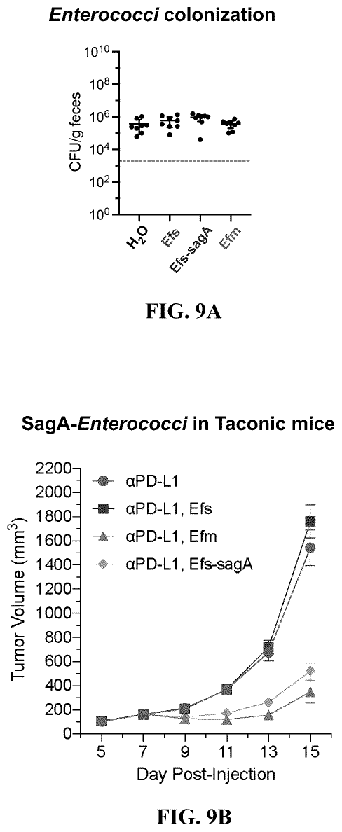

Importantly, many Enterococcus spp. (Efm, Efs, Eds, Ehe, Emi, Egm) are able to colonize the gastrointestinal tract of the mouse (FIG. 8B). This was investigated by determining the CFU/g of feces. Fecal samples were sterilely collected three days after the start of Enterococcus spp. administration. Samples were weighed, resuspended in sterile PBS, homogenized by grinding with sterile pestles, serially diluted in sterile PBS, and then plated by drip assay onto selective BD BBL Enterococcosel agar plates (Fisher Scientific, B12205). Plates were incubated for 24-48 h at 37° C. under ambient atmospheric pressure until colonies formed. Colonies were then manually counted to determine the results found in FIGS. 8B and 9B.

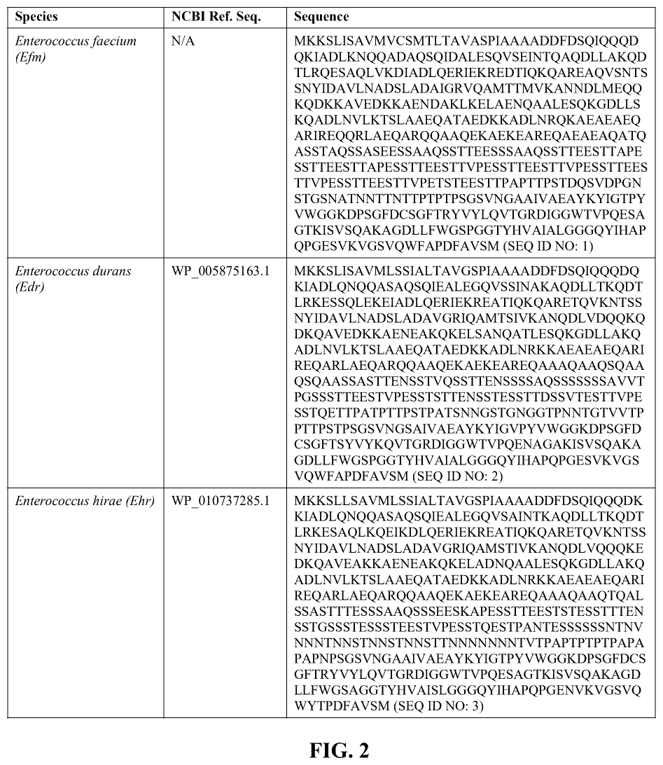

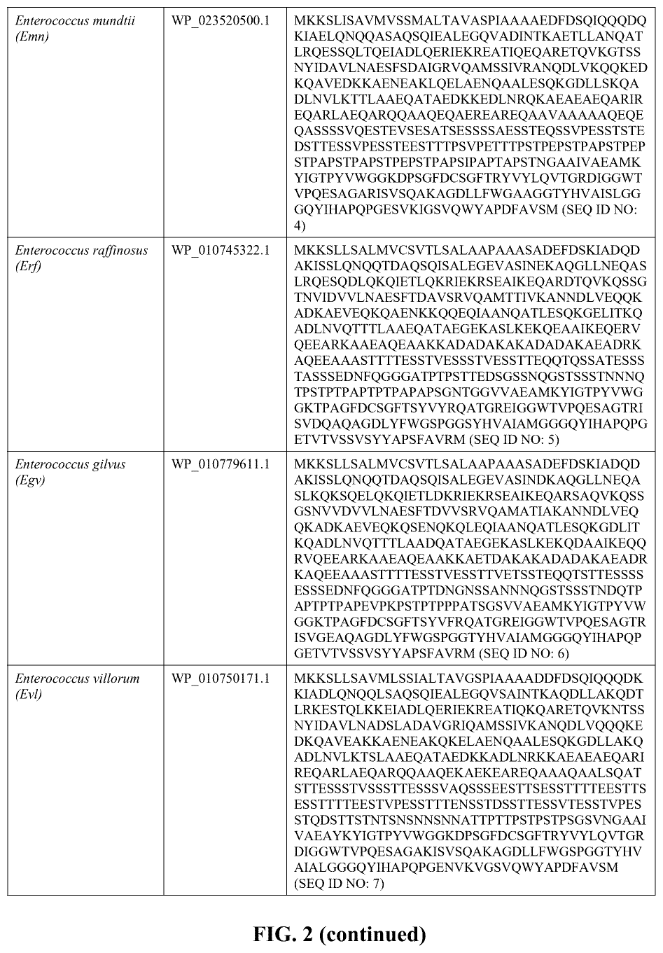

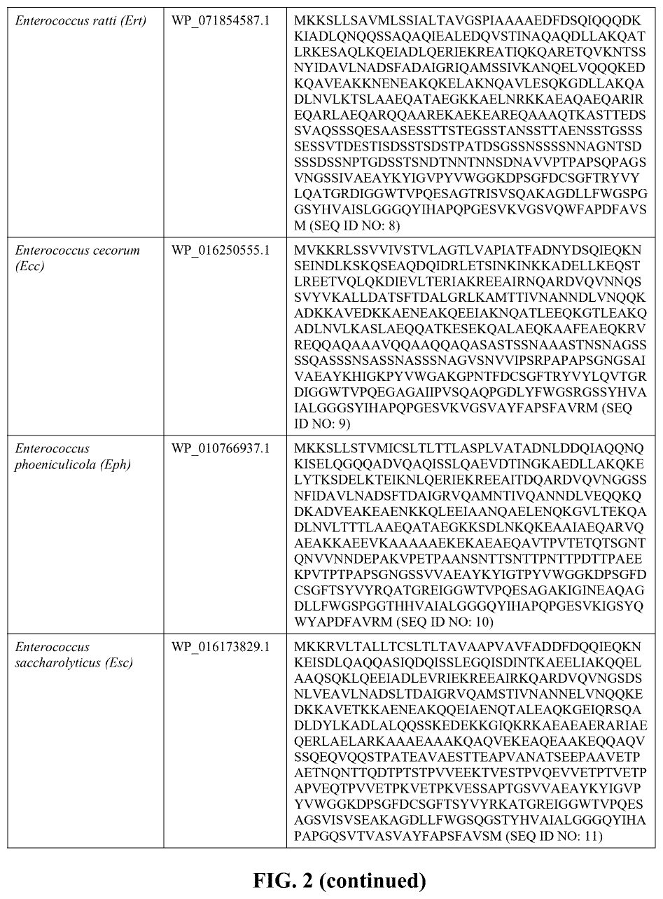

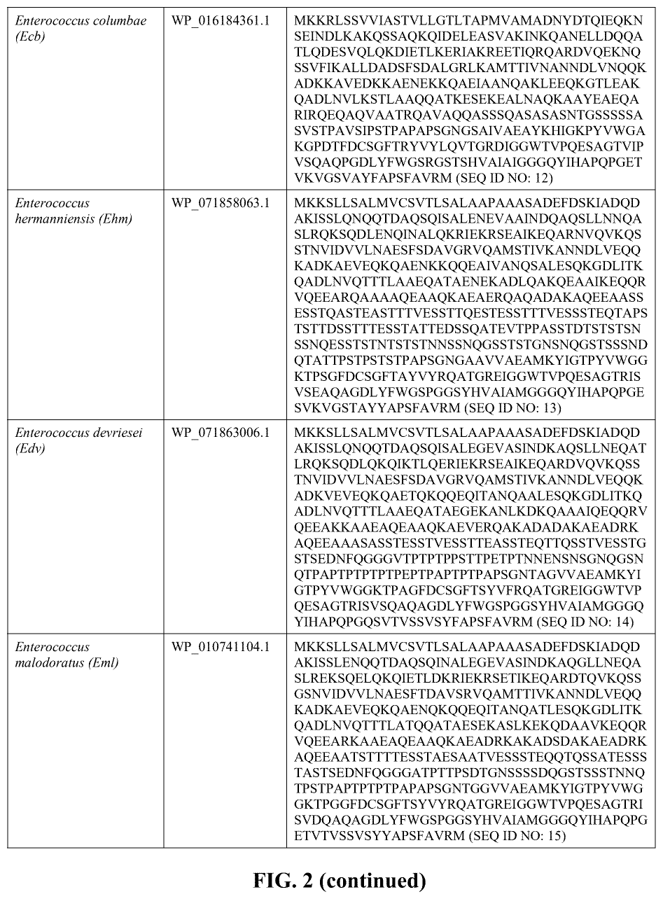

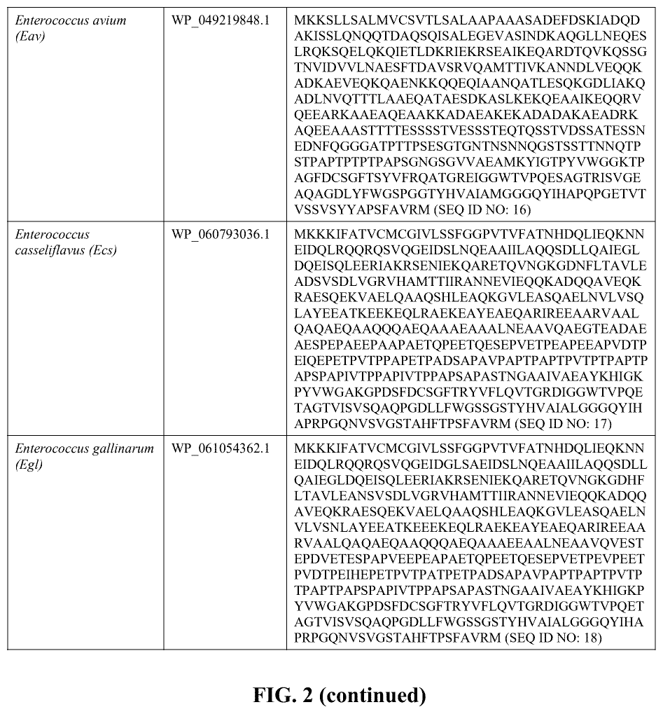

These results indicate that SagA-like NlpC/p60 hydrolases in Enterococci species may be a significant determinant of their ICI enhancing activity. It will be recognized that results presented herein indicate that SagA expression in Enterococci may be involved in enhancing ICI efficacy and that SagA orthologs are also present in the other ICI response-associated Enterococci species (FIGS. 1 and 2). The disclosure thus includes the use of Enterococci species that express SagA orthologs with 80 percent protein identity to SagA-NlpC/p60 domain (FIG. 3), listed above as immunomodulators to prevent pathogen infection, adjuvants for vaccine development or in combination with ICI for cancer therapy.

### Example 3—SagA Expression and Secretion in Lactobacillus plantarum and Lactococcus lactis Confers Protection Against Pathogens

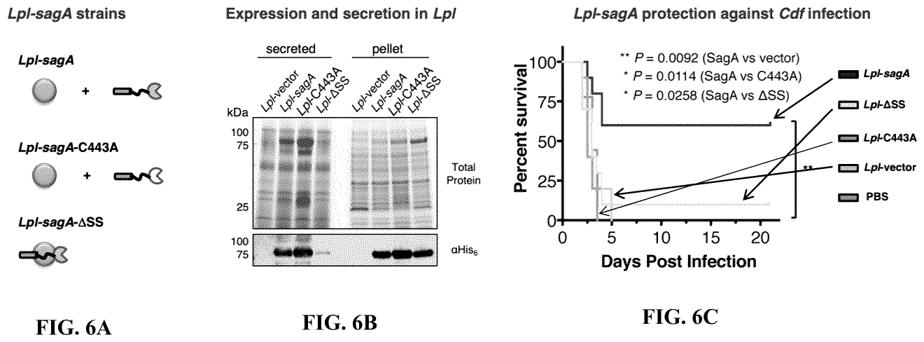

The presently provided discovery of Efm SagA activity, its conservation in other ICI-associated Enterococci strains, and its ability to maintain activity upon transfer to other bacteria as illustrated above provide a unique opportunity to engineer and improve existing probiotics for ICI treatment. As discussed above, it has already been demonstrated that SagA can be expressed in the probiotic species Lactobacillus plantarum (Lpl) and confer protective activity against intestinal pathogens in vivo. Furthermore, we recently demonstrated that SagA secretion and catalytic activity are required for this protective activity in vivo. In a murine model of Clostridium difficile infection, Lpl strains expressing SagA signal sequence (Lpl-ΔSS) and active site (Lpl-C443A) mutants were inactive compared to Lpl expressing wild-type SagA (Lpl-sagA, FIG. 6C).

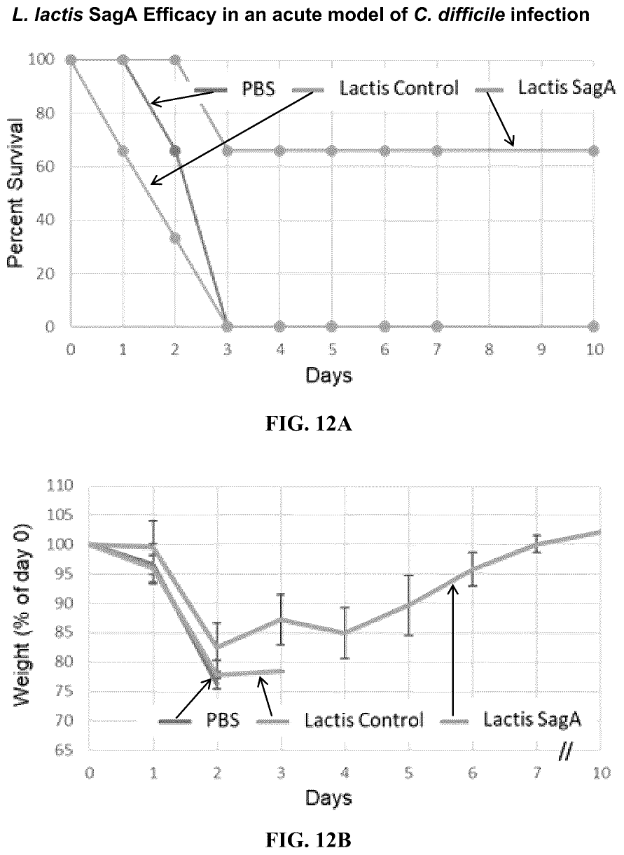

To test the efficacy of L. lactis SagA in an acute model of C. difficile infection, C57BL/6J mice 8 weeks of age were purchased from the Jackson Laboratory and gavaged with the AMNV (4 mg ampicillin, 2 mg metronidazole, 4 mg neomycin, 2 mg vancomycin) antibiotic cocktail daily for 7 days before receiving an oral administration of clindamycin (10 mg/kg). Two days later, mice (8/group) were orally administered one of the following: vehicle phosphate-buffered saline (PBS), 1×10⁹ CFU of a negative control strain of L. lactis (Lactis Control), or 1×10⁹ CFU of L. lactis expressing SagA (Lactis SagA). Mice were then infected with C. difficile 30 hours after treatment. Weight loss was monitored before and during infection for up to 10 days. Mice were euthanized when they reached 80% baseline weight or when they appeared hunched or moribund, whichever occurred first. As shown in the FIG. 12A, L. lactis SagA protected mice from infection compared to the PBS-treated (vehicle control) animals or Lactis Control-treated animals, which all died within 3 days of the C. difficile initial infection. L. lactis SagA treated animals showed some weight loss initially but then began regaining weight after day 3 and by day 10 were back to original weight levels (FIG. 12B). Thus, L. lactis expressing heterologous SagA effectively protected mice against C. difficile infection.

These results support a variety of uses of SagA secretion and extracellular cleavage of peptidoglycan fragments in vivo, and further demonstrate SagA-expression and activity is sufficient to improve ICI efficacy in the B16/F10 melanoma model described in Example 1. Further, as discussed in Example 2, the disclosure includes use of other Enterococci SagA orthologs and SagA variant for heterologous expression and secretion in the probiotics. In addition to the naturally occurring variants of SagA, the disclosure includes structural variants such as the SagA-NlpC/p60 domain alone with signal sequence, which is more catalytically active than the purified full-length SagA in vitro as well as rationally engineered SagA variants. While the invention has been described through specific embodiments, routine modifications will be apparent to those skilled in the art and such modifications are intended to be within the scope of the present invention.

### Example 4—SagA Expression can Treat Immunotherapy Non-Responsive Microbiotia In Vivo

In non-limiting embodiments, the disclosure includes assessing, using non-limiting examples of SagA-expressing Enterococci, to assess whether colonization is sufficient to reprogram non-response microbiota for cancer immunotherapy. In certain embodiments, the well-established tumor growth model with B16/F10 syngeneic melanoma cells in SPF-Taconic mice (which have a less responsive microbiota) is used to illustrate certain approaches of this disclosure and an antibiotic mixture was not used to modify the microbiota of SPF-Taconic mice. However, similar to the protocol of Example 1, on the day prior to administration, Enterococcus spp. were inoculated into 4 mL of autoclaved growth medium and grown as overnight cultures, diluted to a ratio of 1:50, grown to late logarithmic phase, and then resuspended in sterile-filtered drinking water. Bacteria were then diluted in two 50-mL aliquots per animal cage. Tubes were provided to the animals ad libitum. As controls, separate cohorts are inoculated with the non-protective species Enterococcus faecalis (Efs, strain OG1RF) or vehicle only. After 24 h, the animals are then subcutaneously injected with cultured B16/F10 melanoma cells. Tumor growth is quantified by digital calipers. Starting on day 9 post-injection, mice are intraperitoneally (i.p.) injected with), such as every two days for four total injections.

Experiments indicated that Efm and Efs-sagA, but not Efs can inhibit tumor growth after anti-PD-L1 treatment in a murine model of melanoma with a less responsive microbiota (FIG. 9B). Efs, Efs-sagA, and Efm are all able to colonize the gastrointestinal tract of the SPF-Taconic mice (FIG. 9A). The methods used to determine colonization are explained in Example 2. The data presented indicate that heterologous expression of SagA can significantly improve anti-PD-L1 therapies in murine models of melanoma with less responsive microbiotas.

### Example 5—Nod2 is Required for SagA-Mediated Anti-Tumor Immunotherapy

In non-limiting embodiments, the disclosure includes assessing, using non-limiting examples of SagA-expressing Enterococci, to assess whether Nod2 is required for SagA-mediated cancer immunotherapy. In certain embodiments, the well-established tumor growth model with B16/F10 syngeneic melanoma cells in NOD2^{-/-} mice (The Jackson Laboratory, 005763) is used to illustrate certain approaches of this disclosure. Similarly to the protocol of Example 1, mice are first treated with an antibiotic (Abx) cocktail. On the day prior to administration, Enterococcus spp. were inoculated into 4 mL of autoclaved growth medium and grown as overnight cultures, diluted to a ratio of 1:50, grown to late logarithmic phase, and then resuspended in sterile-filtered drinking water. Bacteria were then diluted in two 50-mL aliquots per animal cage. Tubes were provided to the animals ad libitum. As controls, separate cohorts are inoculated with the non-protective species Enterococcus faecalis (Efs, strain OG1RF) or vehicle only. After 24 h, the animals are then subcutaneously injected with cultured B16/F10 melanoma cells. Tumor growth is quantified by digital calipers. Starting on day 9 post-injection, mice are intraperitoneally (i.p.) injected with), such as every two days for four total injections.

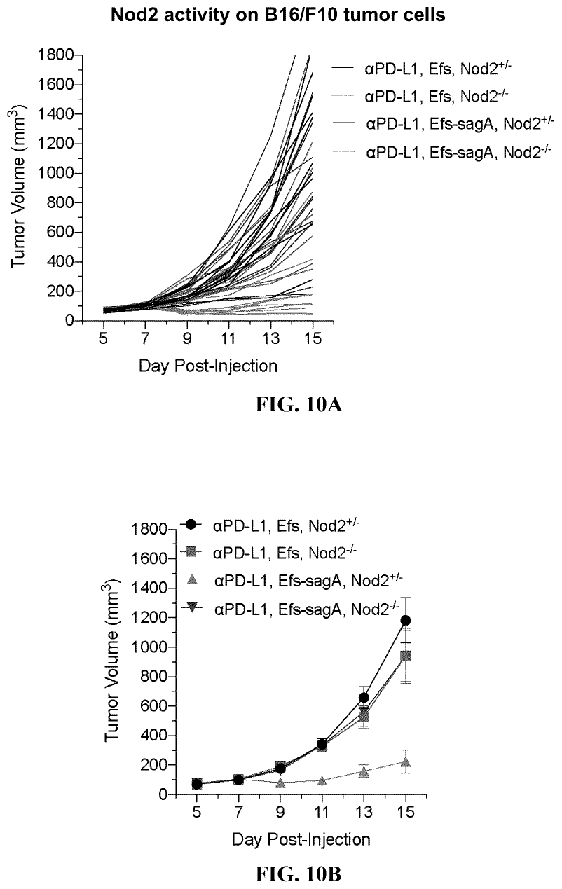

Experiments indicated that neither Efs-sagA nor Efs can inhibit tumor growth after anti-PD-L1 treatment in a NOD2^{-/-} murine model of melanoma (FIGS. 10A and 10B). However, Efs-sagA significantly inhibits tumor growth NOD2^{+/-} murine model of melanoma when compared to Efs-sagA in a NOD2^{-/-} murine model of melanoma (FIGS. 10A and 10B). The data presented indicate that heterologous expression of SagA can significantly improve anti-PD-L1 therapies in murine models of melanoma only if Nod2 is present.

### Example 6—L. lactis Expressing Heterologous SagA Restores Immune Checkpoint Inhibitor Efficacy

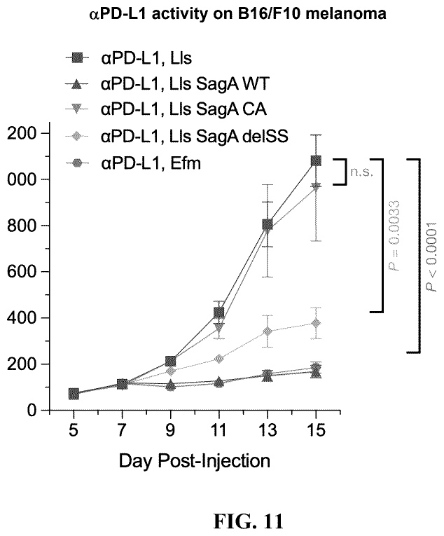

The ability for various, genetically modified L. lactis strains to restore immune checkpoint inhibitor efficacy was tested; the various L. lactis strains have either a wild-type SagA (Lls WT), a catalytically inactive version of SagA with a mutation at residue 384 that abolishes hydrolase activity (Lls CA), or a signal sequence deletion version which eliminates SagA cellular (Lls delSS). Animals were fed 10⁹ CFU/ml of each strain in the context of the B16/F10 murine tumor model methods discussed in Example 1. For this study, tumor growth was quantified by digital calipers every other day on days 5-15. All tumor growth experiments were statistically analyzed by linear mixed modeling. While tumors grew at a rapid and consistent rate for the group treated with anti-PD-L1 and L. lactis, the group treated with L. lactis expressing WT SagA (Lls WT) demonstrated profound anti-tumor activity, which was similar to E. faecium (which naturally expresses SagA) when combined with anti-PD-L1 treatment (FIG. 11). In contrast, the catalytically inactive L. lactis SagA (Lls CA) demonstrated no anti-tumor activity, and the L. lactis line expressing the deleted signal peptide sequence and deficient for secretion of SagA (Lls delSS) demonstrated a statistically significant anti-tumor effect but not as robust as Lls WT (FIG. 11). Thus, the results demonstrated the ability for SagA expressed by L. lactis to restore checkpoint inhibitor efficacy.

While the invention has been described through specific embodiments, routine modifications will be apparent to those skilled in the art and such modifications are intended to be within the scope of the present invention.

We claim:

1. A method of treating an individual with cancer comprising introducing into an individual in need thereof modified bacteria comprising a heterologous secreted antigen A (SagA) or an ortholog of SagA, said heterologous SagA having at least 80% sequence identity to SEQ ID NO: 1 and said ortholog having a NlpC/p60 hydrolase domain having at least 80% sequence identity to SEQ ID NO: 19, wherein the modified bacteria express the heterologous secreted antigen A (SagA), the method further comprising administering to the individual an immunotherapy regimen.

2. The method of claim 1, wherein the modified bacteria are introduced as a component of a food product, a probiotic formulation, or a pharmaceutical formulation.

3. The method of claim 1, wherein the individual has been diagnosed with cancer or is at risk of developing a relapse of cancer.

4. The method of claim 1, wherein the individual is resistant to treatment with the immune checkpoint inhibitor.

5. The method of claim 1, wherein the immunotherapy regimen is T-cell transfer therapy, antibody targeting of cancer, a cancer vaccine, delivery of immune system modulators, and/or administration of immune checkpoint inhibitors.

6. The method of claim 5, wherein the immune checkpoint inhibitors are nivolumab, pembrolizumab, durvalumab, atezolizumab, avelumab, ipilimumab, tremelimumab or combinations thereof.

7. The method of claim 1, wherein the modified bacteria are gram positive or gram negative bacteria.

8. The method of claim 7, wherein the modified bacteria are gram positive bacteria and are selected from Lactobacillus plantarum, Lactobacillus casei, Lactobacillus acidophilus, Lactobacillus salivarius, Lactobacillus reuteri, Lactococcus lactis or Bifidobacterium longum.

9. The method of claim 1, wherein the heterologous SagA is Enterococcus faecium SagA, or a SagA ortholog from: Enterococcus durans, Enterococcus hirae, Enterococcus mundtii, Enterococcus raffinosus, Enterococcus gilvus, Enterococcus villorum, Enterococcus ratti, Enterococcus cecorum, Enterococcus phoeniculicola, Enterococcus saccharolyticus, Enterococcus columbae, Enterococcus hermanniensis, Enterococcus devriesei, Enterococcus malodoratus, Enterococcus avium, Enterococcus casseliflavus, or Enterococcus gallinarum.

10. The method of claim 1, wherein the bacteria comprises an ortholog of the heterologous SagA that has 80-89% sequence identity to SEQ ID NO: 19.

11. The method of claim 1, wherein the bacteria comprises a heterologous SagA that has at least 80-89% least sequence identity to SEQ ID NO: 1.

12. A method of treating an individual with a bacterial infection comprising introducing into an individual in need thereof modified bacteria comprising a heterologous secreted antigen A (SagA) or an ortholog of SagA, said heterologous SagA having at least 80% sequence identity to SEQ ID NO: 1 and said ortholog having a NlpC/p60 hydrolase domain having at least 80% sequence identity to SEQ ID NO: 19, and/or one or more bacteria that endogenously express SagA, the method further optionally comprising administering to the individual a vaccine, and/or an immune checkpoint inhibitor.

13. The method of claim 12, further comprising administering the vaccine and/or the immune checkpoint inhibitor.

14. The method of claim 12, wherein the modified bacteria are gram positive or gram negative bacteria.

15. The method of claim 14, wherein the modified bacteria are gram positive bacteria and are selected from Lactobacillus plantarum, Lactobacillus casei, Lactobacillus acidophilus, Lactobacillus salivarius, Lactobacillus reuteri, Lactococcus lactis or Bifidobacterium longum.

16. The method of claim 12, wherein the heterologous SagA is Enterococcus faecium SagA, or a SagA ortholog from: Enterococcus durans, Enterococcus hirae, Enterococcus mundtii, Enterococcus raffinosus, Enterococcus gilvus, Enterococcus villorum, Enterococcus ratti, Enterococcus cecorum, Enterococcus phoeniculicola, Enterococcus saccharolyticus, Enterococcus columbae, Enterococcus hermanniensis, Enterococcus devriesei, Enterococcus malodoratus, Enterococcus avium, Enterococcus casseliflavus, or Enterococcus gallinarum.

17. The method of claim 12, wherein the bacteria comprises an ortholog of the heterologous SagA that has 80-89% sequence identity to SEQ ID NO: 19.

18. The method of claim 12, wherein the bacteria comprises a heterologous SagA that has at least 80-89% least sequence identity to SEQ ID NO: 1.
